(19) United States
(12) Patent Application Publication
(10) Pub. No.: US 2014/0235913 A1
(43) Pub. Date: Aug. 21, 2014

(54) PROCESS FOR METATHESIS OF OLEFINS OBTAINED FROM FISCHER-TROPSCH FRACTIONS USING A RUTHENIUM COMPLEX COMPRISING A SYMMETRIC N-HETEROCYCLIC DIAMINOCARBENE

(71) Applicant: IFP Energies nouvelles, Rueil-Malmaison Cedex (FR)

(72) Inventors: Helene OLIVIER-BOURBIGOU, Saint Genis-Laval (FR); Mikael BERTHOD, Lyon (FR)

(73) Assignee: IFP ENERGIES NOUVELLES, Rueil-Malmaison Cedex (FR)

(21) Appl. No.: 14/186,300

(22) Filed: Feb. 21, 2014

(30) Foreign Application Priority Data
Feb. 21, 2013 (FR) 13/51.511

(51) Int. Cl. C07C 6/04

(52) U.S. Cl. CPC C07C 6/04
USPC 585/645

(57) ABSTRACT

This invention describes a process for metathesis of olefins from feedstocks obtained from the Fischer-Tropsch process, using as catalyst a ruthenium indenylidene complex comprising a saturated or unsaturated, symmetric N-heterocyclic carbene.

### FIELD OF THE INVENTION

This invention relates to the metathesis of olefins from feedstocks obtained from the Fischer-Tropsch process, which is a catalytic reaction for transformation of identical or different olefins consisting in exchanging the alkylidine groups of the starting olefins to form new olefins.

### TERMINOLOGY

N-Heterocyclic diaminocarbenes or NHC-heterocyclic carbenes (N-heterocyclic carbene in English) are defined as ligands of the imidazolidine-2-ylidene (saturated heterocyclic NHC) type and ligands of the imidazoline-2-ylidine (unsaturated heterocyclic NHC) type.

Symmetric N-heterocyclic diaminocarbenes are defined as N-heterocyclic diaminocarbenes that carry identical carbon-containing groups on the nitrogen atoms.

### PRIOR ART

The metathesis reaction has become an important tool for forming carbon-carbon bonds. It is implemented in the fields of petrochemistry, polymers, oleochemistry, and fine chemistry. The isolated carbenic complexes based on ruthenium have been described for catalyzing this reaction (Chem. Rev. 2010, 110, 1746-1787).

The patent WO 01/46096 describes a process for converting the C4-C10 olefins obtained from a Fischer-Tropsch process into C6-C18 olefins by using a homogeneous catalyst based on ruthenium of the Grubbs type (1^{st} generation) of the formula RuCl₂(PCy₃)₂(CHPh) with an improved selectivity relative to the heterogeneous catalysts known to one skilled in the art.

The patent WO2007/075427 describes a ruthenium complex that carries an N-heterocyclic carbene with 5 NHC-type members in which one of the nitrogen atoms is substituted by a phenyl group that contains a hydrogen in the ortho position and that is substituted in the prime ortho position. These complexes are used for catalyzing the metathesis of olefins by cycle closing.

The ruthenium (Ru) complexes comprising an N-heterocyclic diaminocarbene ligand with 5 dissymmetric NHC-type members, i.e., carrying non-identical carbon-containing groups, have been described by Blechert (Organometallics, 2006, 25, 25-28 and Dalton Trans. 2012, 41, 8215-8225). A wide variety of ruthenium-based catalysts is described, but each of these catalysts is designed to be applied to a very specific metathesis reaction. Their transposition to another metathesis reaction is not obvious.

The patent WO01/46096 describes a metathesis process for converting C4-C10 short olefins that are primarily alpha-olefins and whose optional branching is positioned with two at least double-bond carbon atoms, able to be derived from a Fischer-Tropsch process, into longer C8-C18 olefins by using homogeneous complexes, primarily based on the Grubbs-type ruthenium (1^{st} generation) of the formula RuCl₂(PCy₃)₂(CHPh) with an improved selectivity relative to the heterogeneous catalysts that are known to one skilled in the art. In this patent, the effect of poisons of certain oxidized compounds contained in the Fischer-Tropsch feedstocks on their conversion by metathesis catalyzed by the Grubbs complex is also described.

It is known that the oxidized derivatives change the behavior of metathesis catalysts (see the review A. by Klerk Green Chem. 2008, Vol. 10, No. 12, pp. 1237-1344).

In a surprising way, it was found that the activity by metathesis and the selectivity of the catalyst with ruthenium indenylidene comprising a saturated or unsaturated symmetric N-heterocyclic (NHC) carbenic ligand is maintained and even improved in a process for metathesis of olefins that is obtained from a Fischer-Tropsch process and that has 3 to 10 carbon atoms, and this is done despite the presence of the oxidized compounds that are usually known for deactivating or limiting the activity of the metathesis catalysts. The process according to the invention makes it possible to obtain olefins with good selectivity for the internal linear olefins while limiting the production of olefins having a wide distribution of carbon atoms.

Usually, a pretreatment of Fischer-Tropsch feedstocks is carried out to eliminate the oxidized compounds before the implementation of the metathesis reaction. This invention makes it possible to simplify this stage, and even to eliminate it, which has the result of improving the overall economy of the process for transformation of the feedstocks obtained from the Fischer-Tropsch process.

Another advantage of the invention is to improve the selectivity of the reaction for metathesis of olefins of the feedstocks obtained from the Fischer-Tropsch process in such a way as to optimize the desired olefin yield, which has the result of optimizing the separation of products and also of improving the overall economy of the process.

The process according to the invention also makes it possible to obtain a good conversion of the olefins to be transformed that are contained in the feedstocks obtained from the Fischer-Tropsch process, and this is done with very small concentrations of ruthenium.

### SUMMARY OF THE INVENTION

This invention relates to a process for metathesis of olefins from feedstocks obtained from the Fischer-Tropsch process, using as catalyst a ruthenium indenylidene complex of Formula (I) or (II)

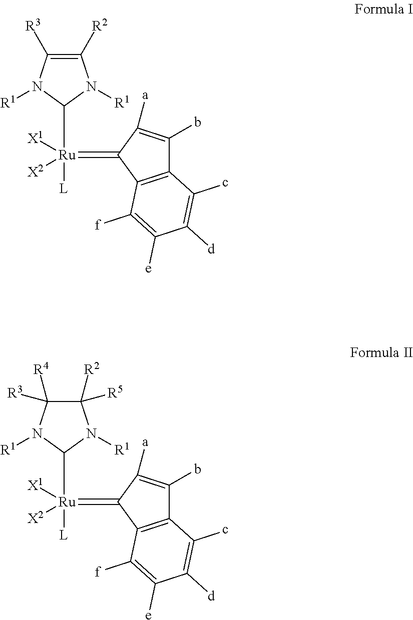

in which:
R¹ is a linear or branched alkyl group, a monocyclic or polycyclic cycloalkyl group, an aromatic monocyclic or bicyclic aryl group, or a linear or branched arylalkyl group that carries an aromatic cycle,
a, b, c, d, e, and f are selected independently from one another in the group that consists of a hydrogen atom, an alkyl group, and a heteroalkyl group,
X₁ and X₂, identical or different, are anionic ligands,
L is a ligand that is an electron donor and uncharged,
R², R³, R⁴, R⁵—identical or different—are hydrogen, halide, alkyl, cycloalkyl, aryl or arylalkyl groups, each being able to be substituted by alkyl, halide, or alkoxy groups or by a phenyl group that is optionally substituted by halide, alkyl, or alkoxy groups.

Advantageously, R¹ is selected from among a linear or branched alkyl group that has 1 to 15 carbon atoms, a monocyclic cycloalkyl group that has 3 to 10 carbon atoms, or a polycyclic cycloalkyl group that has 4 to 18 carbon atoms, an aromatic monocyclic or bicyclic aryl group that has 6 to 20 carbon atoms, or a linear or branched arylalkyl group that carries a monocyclic aromatic cycle that has 7 to 12 carbon atoms. R¹ is preferably selected from the group that consists of phenyl, naphthyl, 2,4,6-trimethylphenyl, 2,6-di-iso-propylphenyl, 3,5-dinitrophenyl, 2,4,6-tris(trifluoromethyl)phenyl, 2,4,6-trichlorophenyl, and hexafluorophenyl, cyclopentyl, cyclohexyl, cycloheptyl, cyclooctyl, cyclodecyl, cyclododecyl, and cyclopentadecyl.

Advantageously, R², R³, R⁴, R⁵ can be identical or different and selected from among:
A hydrogen atom,
A halide,
A linear or branched alkyl group that has 1 to 15 carbon atoms,
A monocyclic cycloalkyl group that has 3 to 10 carbon atoms, or a polycyclic cycloalkyl group that has 4 to 18 carbon atoms,
An aromatic monocyclic or bicyclic aryl group that has 6 to 20 carbon atoms,
A linear or branched arylalkyl group that carries a monocyclic aromatic cycle that has 7 to 12 carbon atoms, with the aliphatic chain comprising 1 or 2 carbon atoms.

X¹ or X² is an anionic ligand that is advantageously selected from among halides, sulfates, alkyl sulfates, aryl sulfates, alkyl sulfonates, aryl sulfonates, alkyl sulfinates, aryl sulfinates, acyls, carbonates, carboxylates, alcoholates, phenolates, amides, and pyrolides, which may or may not be substituted by one or more groups selected from among the alkyl groups having 1 to 12 carbon atoms, the alcoholate groups having 1 to 12 carbon atoms, the aryl groups having 5 to 24 carbon atoms, and the halides, said substituent groups, except for halides, themselves being substituted or not by one or more of the groups that are selected from among the halides, the alkyl groups having 1 to 6 carbon atoms, the alcoholate groups having 1 to 6 carbon atoms, and the aryl groups. Advantageously, X¹ or X² is selected from among the halide ligands, the benzoates, the tosylates, the mesylates, the trifluoromethane-sulfonates, the pyrolides, the CF₃CO₂ trifluoroacetate groups, the CH₃CO₂ acetates, the alcoholates, and the phenolates.

Advantageously, L is a phosphorated ligand of formula PR′₃, in which P is a phosphorus atom and R′ is selected from among the groups R and (OR), in which the groups R are identical or different and are selected from among the following groups: hydrogen, halides, alkyls, cycloalkyls, aryls and aryalkyls, which may or may not be substituted, each of the groups comprising up to 20 carbon atoms, and the substituents of said groups can advantageously be selected from among the halides, the alkyl groups, and the aryl groups that have up to 20 carbon atoms. Preferably, L is a trialkylphosphine or a tricycloalkylphosphine selected from among tricyclohexylphosphine, triisopropylphosphine, and tricyclopentylphosphine; a dialkylphosphine or a dicycloalkylphosphine selected from among dicyclohexylphosphine, dicyclohexylphenylphosphine, di-tert-butylphosphine and di-tert-butylchlorophosphine; or a triarylphosphine selected from among triphenylphosphine, tri(methylphenyl)phosphine, trimesitylphosphine, tri(dimethylphenyl)phosphine, or tri[(trifluoromethyl)phenyl]phosphine.

In one variant, X¹ and X² are identical and are selected from among the chloride or bromide ligands, and L is a tricyclohexylphosphine.

The feedstock according to the invention advantageously comprises linear olefins or linear alpha-olefins that have 3 to 10 carbon atoms.

Preferably, the feedstock also comprises branched olefins and internal olefins or alkanes or oxidized derivatives.

Advantageously, the feedstock is selected from among the fractions that contain more than 30% by weight of olefins that have 4 to 9 carbon atoms, of which more than 70% by weight are alpha-linear, less than 70% by weight alkanes, and less than 10% oxidized compounds.

The quantity of ruthenium complex relative to the linear alpha-olefins expressed in mols is advantageously between 1 and 10,000 ppm.

The process according to the invention is advantageously implemented at a temperature of between 0° C. and 180° C. and at a pressure of between atmospheric pressure and 10 MPa.

### DETAILED DESCRIPTION OF THE INVENTION

The invention relates to a process for metathesis of olefins from feedstocks obtained from the Fischer-Tropsch process using as catalyst a ruthenium indenylidene complex of Formula (I) or (II)

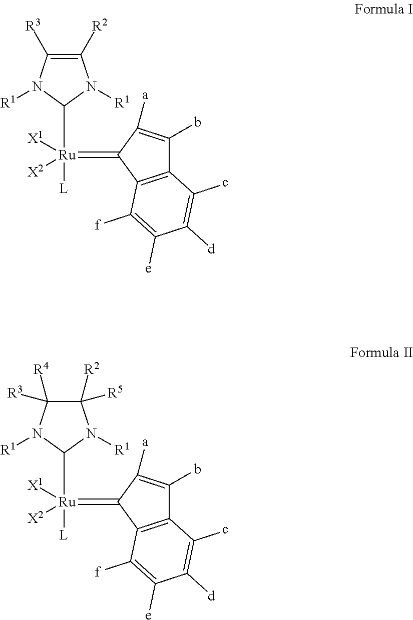

in which:
R¹ is a linear or branched alkyl group, a monocyclic or polycyclic cycloalkyl group, an aromatic monocyclic or bicyclic aryl group, or a linear or branched arylalkyl group that carries an aromatic cycle,
a, b, c, d, e, and f are selected independently from one another in the group that consists of a hydrogen atom, an alkyl group, and a heteroalkyl group,
X₁ and X₂, identical or different, are anionic ligands,
L is a ligand that is an electron donor and uncharged,
R², R³, R⁴, R⁵—identical or different—are hydrogen, halide, alkyl, cycloalkyl, aryl or arylalkyl groups, each being able to be substituted by alkyl, halide, or alkoxy groups or by a phenyl group that is optionally substituted by halide, alkyl, or alkoxy groups.

### The Catalyst

The catalyst that is used in the process according to the invention is a saturated or unsaturated symmetric N-heterocyclic carbenic (NHC) complex that is based on ruthenium.

The catalyst that is used in the process of the invention is a ruthenium alkylidene complex corresponding to Formulas (I) or (II) in which

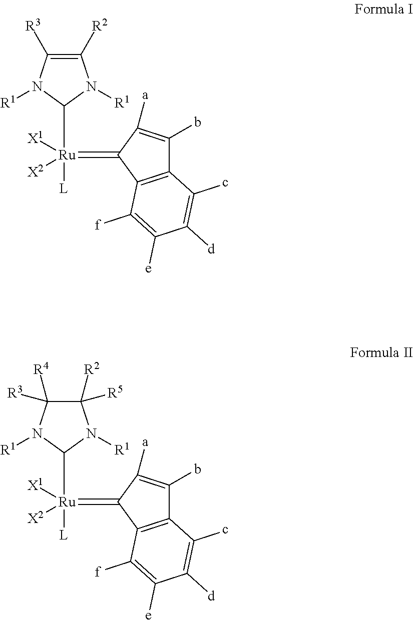

in which:
R¹ is a linear or branched alkyl group, a monocyclic or polycyclic cycloalkyl group, an aromatic monocyclic or bicyclic aryl group, or a linear or branched arylalkyl group that carries an aromatic cycle,
a, b, c, d, e, and f are selected independently from one another in the group that consists of a hydrogen atom, an alkyl group, and a heteroalkyl group,
X₁ and X₂, identical or different, are anionic ligands,
L is a ligand that is an electron donor and uncharged,
R², R³, R⁴, R⁵—identical or different—are hydrogen, halide, alkyl, cycloalkyl, aryl or arylalkyl groups, each being able to be substituted by alkyl, halide, or alkoxy groups or by a phenyl group that is optionally substituted by halide, alkyl, or alkoxy groups.

In terms of this invention, the substituents R², R³, R⁴, R⁵—identical or different—are selected from the group that consists of a hydrogen atom, halides, or alkyl, cycloalkyl, aryl or arylalkyl groups, each being able to be substituted by alkyl, halide, or alkoxy groups or by a phenyl group that is optionally substituted by halide, alkyl, or alkoxy groups.

For R¹, R², R³, R⁴ and R⁵, “alkyl” is defined as a linear or branched hydrocarbon chain that has 1 to 15 carbon atoms, preferably 1 to 10, and even more preferably 1 to 4. Preferred alkyl groups are advantageously selected from among the methyl, ethyl, propyl, isopropyl, butyl, isobutyl and tert-butyl groups.

For R², R³, R⁴, and R⁵, an “alkoxy” substituent is defined as an alkyl-O— group in which the term alkyl has the meaning provided above. Preferred examples of alkoxy substituents are the methoxy or ethoxy groups.

For R¹, R², R³, R⁴, and R⁵, “cycloalkyl” is defined as a cyclic or monocyclic hydrocarbon-containing group that preferably has 3 to 10 carbon atoms, in particular a cyclopentyl or cyclohexyl group, or a polycyclic (bicyclic or tricyclic) group that has 4 to 18 carbon atoms, in particular adamantyl or norbornyl.

For R¹, R², R³, R⁴, and R⁵, “aryl” is defined as an aromatic monocyclic or polycyclic group—preferably a monocyclic or bicyclic group—that has 6 to 20 carbon atoms, preferably mesityl, phenyl or naphthyl. When the group is polycyclic, i.e., it comprises more than one cyclic core, the cyclic cores can be condensed two by two or attached two by two by bonds.

For R¹, R², R³, R⁴, and R⁵, “arylalkyl” or “aralkyl” is defined as a linear or branched hydrocarbon-containing group that carries a monocyclic aromatic cycle that has 7 to 12 carbon atoms, with the aliphatic chain comprising 1 or 2 carbon atoms. A preferred arylalkyl or aralkyl group is the benzyl group.

R², R³, R⁴, and R⁵ can be identical or different and can be selected from among:
A hydrogen atom,
A halide,
A linear or branched alkyl group that has 1 to 15 carbon atoms,
A monocyclic cycloalkyl group that has 3 to 10 carbon atoms, or a polycyclic cycloalkyl group that has 4 to 18 carbon atoms,
An aromatic monocyclic or bicyclic aryl group that has 6 to 20 carbon atoms,
A linear or branched arylalkyl group that carries a monocyclic aromatic cycle that has 7 to 12 carbon atoms, with the aliphatic chain comprising 1 or 2 carbon atoms.

R¹ is advantageously selected from among a linear or branched alkyl group that has 1 to 15 carbon atoms, a monocyclic cycloalkyl group that has 3 to 10 carbon atoms, or a polycyclic cycloalkyl group that has 4 to 18 carbon atoms, with an aromatic monocyclic or bicyclic aryl group having 6 to 20 carbon atoms, and a linear or branched arylalkyl group that carries a monocyclic aromatic cycle having 7 to 12 carbon atoms. Preferably, R1 is preferably selected from among the mesityl, phenyl or naphthyl groups.

### The Ligands X¹ and X²

X¹ or X² is an anionic ligand that can be selected from among halides, sulfates, alkyl sulfates, aryl sulfates, alkyl sulfonates, aryl sulfonates, alkyl sulfinates, aryl sulfinates, acyls, carbonates, carboxylates, alcoholates, phenolates, amides, and pyrolides, which may or may not be substituted by one or more groups selected from among the alkyl groups having 1 to 12 carbon atoms, the alcoholate groups having 1 to 12 carbon atoms, the aryl groups having 5 to 24 carbon atoms, and the halides, said substituent groups, except for halides, themselves being substituted or not by one or more of the groups that are selected from among the halides, the alkyl groups having 1 to 6 carbon atoms, the alcoholate groups having 1 to 6 carbon atoms, and the aryl groups.

Preferably, X¹ or X² is selected from among the halide ligands, the benzoates, the tosylates, the mesylates, the trifluoromethane-sulfonates, the pyrolides, the CF₃CO₂ trifluoroacetate groups, the CH₃CO₂ acetates, the alcoholates, and the phenolates.

In a preferred manner, the anionic ligands X¹ or X² are selected from among the halide ligands, and in a very preferred manner, the ligands X¹ and X² are identical and are chlorides or bromides.

### The Ligand L

According to the invention, L is an uncharged electron-donor ligand.

In one embodiment, the indenylidene group and the ligand L can be combined within the same chemical entity.

In one embodiment, L is a phosphorated ligand of formula PR′₃, in which P is a phosphorus atom and R′ is selected from among the groups R and (OR), in which the groups R are identical or different and are selected from among the following groups: hydrogen, halides, alkyls, cycloalkyls, aryls and arylalkyls, which may or may not be substituted, each of the groups comprising up to 20 carbon atoms, and the substituents of said groups can advantageously be selected from among the halides, the alkyl groups and the aryl groups that have up to 20 carbon atoms.

The phosphorated ligand L of the ruthenium compound is preferably a phosphine, in a preferred manner a trialkylphosphine or a tricycloalkylphosphine that is selected from among tricyclohexylphosphine, triisopropylphosphine and tricyclopentylphosphine; a dialkylphosphine or a dicycloalkylphosphine that is selected from among dicyclohexylphosphine, dicyclohexylphenylphosphine, di-tert-butylphosphine, and di-tert-butylchlorophosphine or a triarylphosphine that is selected from among triphenylphosphine, tri(methylphenyl)phosphine, trimesitylphosphine, tri(dimethylphenyl)phosphine, and tri[(trifluoromethyl)phenyl]phosphine.

In a preferred manner, X¹ and X² are identical and are selected from among the chloride or bromide ligands; L is a tricyclohexylphosphine.

In a preferred manner, the catalyst that is used in this invention corresponds to the following formulas:

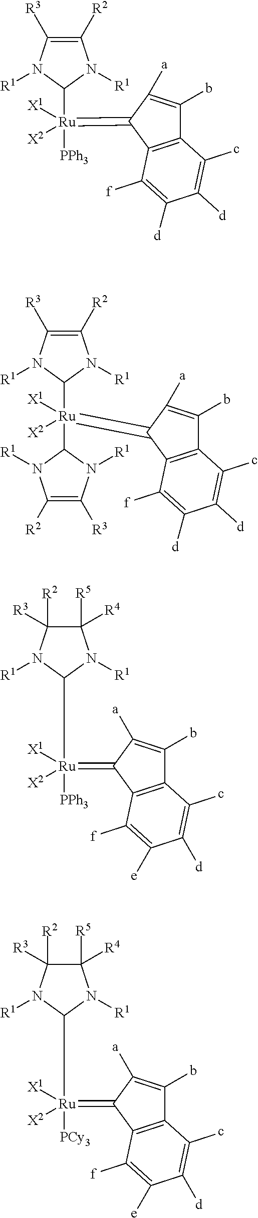

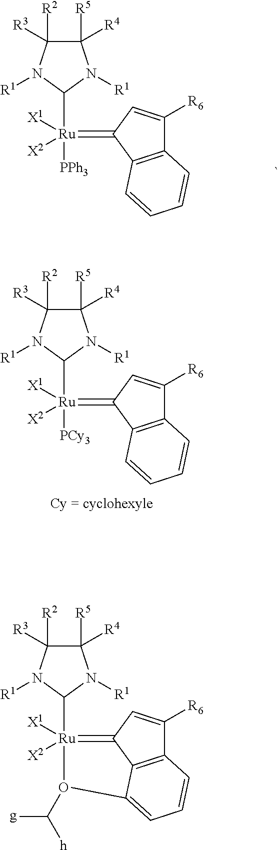

in which:
X¹ and X², identical or different, are as defined above, and preferably selected from among the chloride and bromide ligands.
R³ and R⁴, R⁵, R⁶ are defined as for Formula (I) or (II), and are preferably a hydrogen atom.
R¹, defined as for Formula (I) or (II), is preferably selected from the group that consists of phenyl, naphthyl, 2,4,6-trimethylphenyl, 2,6-di-iso-propylphenyl, 3,5-dinitrophenyl, 2,4,6-tris(trifluoromethyl)phenyl, 2,4,6-trichlorophenyl, and hexafluorophenyl, cyclopentyl, cyclohexyl, cycloheptyl, cyclooctyl, cyclodecyl, cyclododecyl, and cyclopentadecyl.
a, b, c, d, e, f, g, and h are selected independently from one another in the group that consists of a hydrogen atom, an alkyl group, and a heteroalkyl group. g and h optionally can form a cycle.
R⁶ can be a hydrogen or an aryl group that may or may not be substituted. In a preferred manner, R⁶ is a phenyl group that may or may not be substituted.

Preferably, the ligands X¹ and X² are identical and are selected from among the chloride or bromide ligands.

In a preferred manner, L is a tricyclohexylphosphine.

### The Feedstock

The feedstocks that are obtained from the Fischer-Tropsch process are advantageously selected from among the light feedstock fractions, i.e., the fractions that contain for the most part linear olefins and even more advantageously linear alpha-olefins having 3 to 10 carbon atoms.

The feedstocks according to the invention can contain branched olefins and internal olefins. They can also contain alkanes.

The feedstocks according to the invention can contain oxidized derivatives. These oxidized derivatives are advantageously alcohols, aldehydes, ketones, and/or acids. In a preferred way, the oxidized compounds are for the most part alcohols.

In a preferred way, the Fischer-Tropsch fractions according to the invention are selected from among the fractions that contain more than 30% by weight of olefins having 4 to 9 carbon atoms, of which more than 70% are linear alpha-olefins, less than 70% by weight alkanes and less than 10% oxidized compounds.

### Employing the Catalyst

The quantity of catalytic composition used for the metathesis reaction depends on a variety of factors such as the identity of the reagents and of the reaction conditions that are employed. As a result, the necessary quantity of catalytic composition will be defined in an optimal and independent manner for each reaction. However, the quantity of ruthenium complex relative to the olefins, expressed in mols, is preferably between 1 and 10,000 ppm, in a preferred manner between 1 and 200 ppm, and in a particularly preferred manner between 1 and 100 ppm.

### The Process

The process for metathesis of olefins according to the invention can advantageously be carried out in the absence or in the presence of a solvent. If necessary, usable solvents according to the process of the invention can be selected from among the organic solvents, the protic solvents, or water. The solvents that can be used for metathesis according to this invention can, for example, be selected from among the aromatic hydrocarbons such as benzene, toluene, and xylenes; the halogenated aromatic hydrocarbons such as chlorobenzene and dichlorobenzene; the aliphatic hydrocarbons such as pentane, hexane, heptane and cyclohexane; the chlorinated alkanes such as dichloromethane, chloroform, and 1,2-dichloroethane; the ethers such as diethyl ether, and tetrahydrofuran; the alcohols such as methanol and ethanol, or water. A preferred solvent is chlorobenzene.

The combinations of these solvents can also advantageously be used. Any quantity of solvent can advantageously be used, but the use at least of the minimum quantity required for dissolving the compounds of Formula (I) or (II) is preferred, and such a minimum quantity is easily determined by one skilled in the art. The volume of the solvent can be very low relative to the volume of olefin reagents that are used.

The process for metathesis of the olefins according to the invention is advantageously implemented under vigorous stifling to the extent that it makes possible good contact between the reagents (some of which can be gaseous) and said catalytic composition.

The process for metathesis of the olefins according to the invention can advantageously be implemented under an atmosphere of nitrogen or argon, preferably at atmospheric pressure. Generally, a wide range of temperatures can be used. The process for metathesis of the olefins according to the invention is advantageously implemented at a temperature of between 0° C. and 180° C., and preferably between 20° C. and 150° C.

The pressure of the reaction is advantageously between atmospheric pressure and 10 MPa (100 bar) and preferably between atmospheric pressure and 3 MPa (30 bar). If the reagent is gaseous, it is advantageously used in pure form or in a mixture or diluted with an inert paraffin.

The process for metathesis of the olefins according to the invention can be conducted both in a closed system (batch) and in a semi-open system or in a continuous system, and this is done with one or more reaction stages.

Generally, the reaction time or the dwell time in a continuous reaction for the process for metathesis of olefins according to the invention is advantageously from approximately one second to approximately one day, preferably approximately five minutes to approximately 10 hours.

The invention will also be further explained based on the illustrative examples provided below, which demonstrate the advantages of the catalytic compositions and of the process according to the invention.

The examples below illustrate the invention without limiting its scope.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding French Application No. 13/51.511, filed Feb. 21, 2013 are incorporated by reference herein.

### EXAMPLES

### The Fischer-Tropsch Fraction (Denoted FT Fraction Below)

The Fischer-Tropsch fraction that is used contains hydrocarbons that have 4 to 9 carbon atoms. It contains 36% olefins, 60% alkanes, and 4% oxidized compounds. The olefins consist of 1.5% branched olefins, 81% linear alpha-olefins, and 18% internal linear olefins (see Table 1).

TABLE 1
Description of the Fischer-Tropsch Fraction Used in Examples 4-8

| | | | | | | | % | % |
|---|---|---|---|---|---|---|---|---|
| | % C4 | % C5 | % C6 | % C7 | % C8 | % C9 | Alcohol | Total |
| Total | 0.28 | 8.34 | 31.89 | 45.15 | 10.18 | 0.09 | 4.07 | 100 |
| Alkenes | 0.09 | 2.58 | 11.49 | 17.32 | 4.8 | 0.08 | | 36.36 |
| Alkanes | 0.19 | 5.74 | 20.4 | 27.83 | 5.38 | 0.01 | | 59.55 |

### Examples 1-2

### General Protocol of the Metathesis Reaction on the FT Fraction

Approximately exactly [sic] 3,400 mg of freshly distilled FT fraction, filtered on basic alumina and degassed before the test, is injected into a reaction tube under a stream of argon. The heating set-point of the tube is adjusted to 50° C. When the set-point is reached, the catalyst is added in solution in a small quantity of chlorobenzene (≦0.5 ml). This corresponds to the time t=0 of the reaction. At the end of 4 hours, the catalyst is neutralized with several drops of butyl vinyl ether. The analysis of gas phase chromatography then makes it possible for us to determine the conversion of the olefins and to quantify the products that are formed. The performances of the catalyst are defined by:
The selectivity of C10+ olefins (olefins that have 10 and more carbon atoms) according to the following formula:

((the sum of the masses of C10 to C14 olefins)/(the sum of the masses of C4 to C14 olefins))×100.
The conversion of olefins that have 6 carbon atoms according to the following formula:

((the mass of all of the starting C6 olefins—the mass of all of the final C6 olefins)/the mass of all of the starting C6 olefins)×100

The results are summarized in Table 2.

TABLE 2

| | | Selec- |
|---|---|---|
| | Con- | tivity |
| | version | of |
| | of C6 | C10 + |
| | Olefins | Olefins |
| Catalyst | (%) | (%) |
| 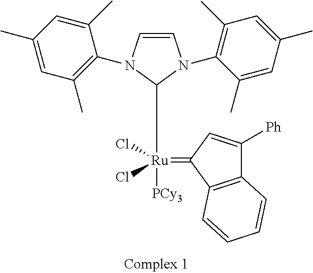 | 56 | 46 |
| 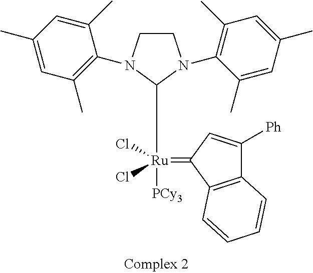 | 70 | 39 |

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Process for metathesis of olefins from feedstocks obtained from the Fischer-Tropsch process, using as catalyst a ruthenium indenylidene complex of Formula (I) or (II) 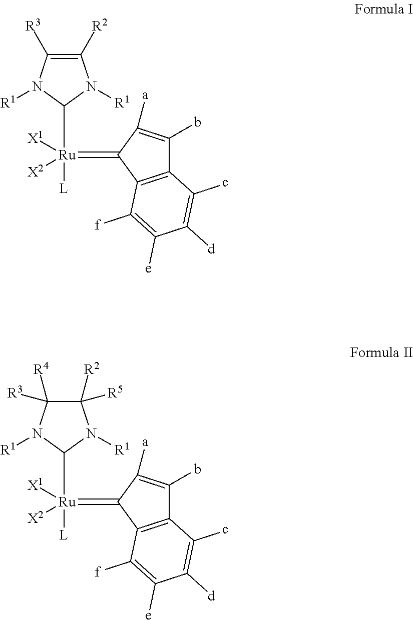
in which:
R¹ is a linear or branched alkyl group, a monocyclic or polycyclic cycloalkyl group, an aromatic monocyclic or bicyclic aryl group, or a linear or branched arylalkyl group that carries an aromatic cycle,
a, b, c, d, e, and f are selected independently from one another in the group that consists of a hydrogen atom, an alkyl group, and a heteroalkyl group,
X₁ and X₂, identical or different, are anionic ligands,
L is a ligand that is an electron donor and uncharged,
R², R³, R⁴, R⁵—identical or different—are hydrogen, halide, alkyl, cycloalkyl, aryl or arylalkyl groups, each being able to be substituted by alkyl, halide, or alkoxy groups or by a phenyl group that is optionally substituted by halide, alkyl, or alkoxy groups.

2. Process according to claim 1, in which R¹ is selected from among a linear or branched alkyl group that has 1 to 15 carbon atoms, a monocyclic cycloalkyl group that has 3 to 10 carbon atoms, or a polycyclic cycloalkyl group that has 4 to 18 carbon atoms, an aromatic monocyclic or bicyclic aryl group that has 6 to 20 carbon atoms, or a linear or branched arylalkyl group that carries a monocyclic aromatic cycle that has 7 to 12 carbon atoms.

3. Process according to claim 1, in which R¹ is preferably selected from the group that consists of phenyl, naphthyl, 2,4,6-trimethylphenyl, 2,6-di-iso-propylphenyl, 3,5-dinitrophenyl, 2,4,6-tris(trifluoromethyl)phenyl, 2,4,6-trichlorophenyl, and hexafluorophenyl, cyclopentyl, cyclohexyl, cycloheptyl, cyclooctyl, cyclodecyl, cyclododecyl, and cyclopentadecyl.

4. Process according to claim 1, in which R², R³, R⁴, R⁵ can be identical or different and selected from among:
A hydrogen atom,
A halide,
A linear or branched alkyl group that has 1 to 15 carbon atoms,
A monocyclic cycloalkyl group that has 3 to 10 carbon atoms, or a polycyclic cycloalkyl group that has 4 to 18 carbon atoms,
An aromatic monocyclic or bicyclic aryl group that has 6 to 20 carbon atoms,
A linear or branched arylalkyl group that carries a monocyclic aromatic cycle that has 7 to 12 carbon atoms, with the aliphatic chain comprising 1 or 2 carbon atoms.

5. Process according to claim 1, in which X¹ or X² is an anionic ligand that is selected from among halides, sulfates, alkyl sulfates, aryl sulfates, alkyl sulfonates, aryl sulfonates, alkyl sulfinates, aryl sulfinates, acyls, carbonates, carboxylates, alcoholates, phenolates, amides, and pyrolides, which may or may not be substituted by one or more groups selected from among the alkyl groups having 1 to 12 carbon atoms, the alcoholate groups having 1 to 12 carbon atoms, the aryl groups having 5 to 24 carbon atoms, and the halides, said substituent groups, except for halides, themselves being substituted or not by one or more of the groups that are selected from among the halides, the alkyl groups having 1 to 6 carbon atoms, the alcoholate groups having 1 to 6 carbon atoms, and the aryl groups.

6. Process according to claim 1, in which X¹ or X² is selected from among the halide ligands, the benzoates, the tosylates, the mesylates, the trifluoromethane-sulfonates, the pyrolides, the CF₃CO₂ trifluoroacetate groups, the CH₃CO₂ acetates, the alcoholates, and the phenolates.

7. Process according to claim 1, in which L is a phosphorated ligand of formula PR′₃, in which P is a phosphorus atom, and R′ is selected from among the groups R and (OR), in which the groups R are identical or different and are selected from among the following groups: hydrogen, halides, alkyls, cycloalkyls, aryls and aryalkyls, which may or may not be substituted, each of the groups comprising up to 20 carbon atoms, and the substituents of said groups can advantageously be selected from among the halides, the alkyl groups and the aryl groups having up to 20 carbon atoms.

8. Process according to claim 7, in which L is a trialkylphosphine or a tricycloalkylphosphine selected from among tricyclohexylphosphine, triisopropylphosphine, and tricyclopentylphosphine, a dialkylphosphine or a dicycloalkylphosphine selected from among dicyclohexylphosphine, dicyclohexylphenylphosphine, di-tert-butylphosphine and the di-tert-butylchlorophosphine, or a triarylphosphine selected from among triphenylphosphine, tri(methylphenyl)phosphine, trimesitylphosphine, tri(dimethylphenyl)phosphine, or tri[(trifluoromethyl)phenyl]phosphine.

9. Process according to claim 8, in which the ligands X¹ and X² are identical and are selected from among the chloride or bromide ligands, and L is a tricyclohexylphosphine.

10. Process according to claim 1, in which the feedstock comprises linear olefins or linear alpha-olefins that have 3 to 10 carbon atoms.

11. Process according to claim 10, in which the feedstock also comprises branched olefins and internal olefins or alkanes or oxidized derivatives.

12. Process according to claim 1, in which the feedstock is selected from among the fractions that contain more than 30% by weight of olefins that have 4 to 9 carbon atoms of which more than 70% by weight are linear alpha-olefins, less than 70% by weight alkanes, and less than 10% oxidized compounds.

13. Process according to claim 1, in which the quantity of ruthenium complex relative to the linear alpha-olefins, expressed in mols, is between 1 and 10,000 ppm.

14. Process according to claim 1, implemented at a temperature of between 0° C. and 180° C. and at a pressure of between atmospheric pressure and 10 MPa.
